(19) United States
(12) Patent Application Publication
(10) Pub. No.: US 2015/0320929 A1
(43) Pub. Date: Nov. 12, 2015

(54) PORTABLE INTRAVENOUS (IV) BACKPACK

(71) Applicant: Kylie Simonds, Naugatuck, CT (US)

(72) Inventor: Kylie Simonds, Naugatuck, CT (US)

(21) Appl. No.: 14/662,460

(22) Filed: Mar. 19, 2015

Related U.S. Application Data
(60) Provisional application No. 61/991,023, filed on May 9, 2014.

(51) Int. Cl. A61M 5/14

(52) U.S. Cl. CPC A61M 5/1415; A61M 5/1417; A61M 2005/1416

(57) ABSTRACT

A portable IV backpack compromising a bag having an interior storage space, shoulder straps, a support rod for an IV pouch containing medicinal fluid. The support rod is secured to the bag and projects upwardly above the bag when the backpack is carried on the back of a patient. An upper end of the projecting rod supports a suspended IV pouch and associated tubing for delivery of medicinal fluid from the pouch to the patient. The bag also has an associated holder for a controller that regulates the amount of medicinal fluid delivered to the patient. A shield is connected to the upper end of the support rod to protect the suspended IV pouch.

### CROSS REFERENCE TO RELATED APPLICATIONS

This application is entitled to the benefit of and incorporates by reference essential subject matter disclosed in Provisional Patent Application Ser. No. 61/991,023 filed on May 9, 2014.

### FIELD OF THE INVENTION

The present invention relates to portable intravenous backpacks.

### BACKGROUND OF THE INVENTION

Intravenous (“IV”) therapy is the infusion of a liquid based medication, or other substance, directly into a vein of a patient. An IV delivery system is often used to facilitate intravenous therapy. Patients undergoing IV therapy treatments must be connected to an IV delivery system for extended periods of time. However, most prior art IV delivery systems are not conducive to patient mobility. A basic IV delivery system includes a pouch, associated tubing, a controller and a pole/stand on wheels. The pouch contains medicinal fluid and is suspended from the top of the wheeled pole/stand, typically at or above shoulder height. The controller is mounted to the wheeled pole/stand, typically at chest height. The pouch is connected to the controller via the associated tubing and the controller in turn is further connected to a patient via the associated tubing. Because the pouch is suspended at the top of the wheeled pole/stand, gravity causes the medicinal fluid to flow from the pouch to the controller and, the controller pumps or regulates the flow of medicinal fluid from the controller to the patient.

In many prior art IV delivery systems, the pole/stand is often fitted with wheels that provide for the pole/stand to be moved along a floor or other surface. However, such prior art IV delivery systems limit the patient's mobility as the wheels often require the floor or other surface to be smooth in order to properly facilitate the movement of the pole/stand with the patient. For example, it is difficult for a patient undergoing IV therapy with an IV delivery system that utilizes a wheeled pole/stand to walk across grass, hilly terrain, a rocky path, and/or other rough and/or uneven surfaces.

Patients undergoing treatment may also be in a weakened state due, for example to chemotherapy, and the pole/stand further limits the patient's mobility. Further, in many prior art IV delivery systems, the wheeled pole/stands are often larger than and detached from the patient, particularly pediatric patients, thereby further restricting patient mobility. Moreover, it is also difficult, if not impractical for a patient using an IV delivery system with a wheeled pole/stand to disassemble and re-assemble the pole/stand in order to travel from one place to another in most passenger vehicles during a treatment session.

Additionally, for prior art IV delivery systems in which the controller utilizes a pump, as opposed to gravitational force, to cause the medicinal fluid to flow from the pouch to the patient, the pump is often mounted on the wheeled pole/stand along with the rest of the controller. As most controllers and pumps require electrical power, the prior art IV delivery systems require either a battery that needs a mounting point, or the patients must remain confined to areas near electrical power outlets; accordingly, patient mobility is further restricted.

Several prior art systems have attempted to solve the mobility problems associated with IV delivery systems utilizing a wheeled pole/stand. For example, U.S. Pat. No. 5,626,270 to Tseng (“Tseng”) discloses a t-shaped frame worn on the back with an extendable rod for suspending an IV pouch. Similarly, U.S. Pat. No. 3,547,322 to Dawson et al. (“Dawson”) discloses a wearable harness assembly having an IV pouch suspended from a rod.

However, wearable IV delivery systems, like Tseng and Dawson, do not provide sufficient support or protection to the previously mentioned IV delivery system components (pouch, associated tubing, controller with a pump) under aggressive mobility conditions, such as those encountered by a child or an athlete during play. Aggressive mobility significantly increases the likelihood that the pouch will be bumped, punctured, ripped/torn, and/or suffer other forms of damage. For example, patients wearing IV delivery systems, similar to the ones disclosed by Dawson and Tseng, must be mindful to restrict their movements so as to not cause the pouch to become punctured, crushed, and/or squeezed, by brushing or pressing the pouch against other objects. In particular, a child wearing a device similar to the ones disclosed by Dawson and Tseng would not be able to freely crawl on the ground, run around a playground or park, and/or roll on the ground, as this would likely cause the pouch to become punctured by wood chips, stick, rocks, and/or sharp plastic and metal corners. The pouch would also likely become crushed by other children accidentally stepping on the pouch.

Further, controllers and pumps mounted to wearable IV delivery systems, similar to the ones disclosed in Dawson and Tseng, are exposed to weather elements and contamination.

Further still, mounted and exposed components, such as pumps and controllers, cause patients, especially young and/or small children, to feel intimidated, and/or afraid, which exacerbates an already troublesome illness.

Additionally, prior art wearable IV delivery systems, such as the ones disclosed by Dawson and Tseng, do not provide any storage space to house and/or protect additional pouches and/or other medical supplies.

### SUMMARY OF THE INVENTION

The present invention relates to a portable IV backpack compromising a bag having an interior storage space, at least one shoulder strap, and an upwardly projecting support rod for an IV pouch. The shoulder strap is configured to permit the backpack to be carried on the back of a patient during an IV treatment. The support rod is secured to the bag and projects upwardly above the bag when the backpack is carried on the back of the patient. An upper end of the projecting rod is configured to support a suspended IV pouch and associated tubing for delivery of medicinal fluid from the pouch to the patient.

A protective shield is connected to the upper end of the support rod to both stabilize and protect the suspended IV pouch from damage during treatment. The bag also has an associated holder for a controller that dispenses a regulated amount of medicinal fluid from the suspended IV pouch to the patient. The bag may further include a moveable flap configured to cover the exposed controller when the controller is not being accessed to regulate or turn the controller on or off. The moveable flap may be further configured to obscure the controller and associated regulating controls and indicators from view.

Because the IV backpack accommodates all the basic components to facilitate IV therapy (IV pouch, associated tubing and controller) and is configured to be secured to the back of a patient by the shoulder strap, patients undergoing IV therapy via the IV backpack need not be connected to a wheeled IV pole/stand. The mobility of patients undergoing IV therapy via the IV backpack is not restricted to areas having floors with smooth surfaces. Instead, patients undergoing IV therapy via the IV backpack are more easily able to walk across grass, hilly terrain, a rocky path and/or other similar rough and/or uneven surfaces. Further still, the support rod occupies less space than a wheeled pole/stand, therefore, patients undergoing IV therapy via the IV backpack can easily fit into and ride in most passenger vehicles.

Additionally, the IV backpack may accommodate a larger mobile power supply, such as a battery, that provides extended power to the regulated controller. Thus, the mobility of patients undergoing IV therapy via the IV backpack is not confined to areas near electrical power outlets.

Moreover, unlike the prior art wearable IV delivery systems, similar to those disclosed by Tseng and Dawson, the shield connected to the upper end of the support rod protects the suspended IV pouch from becoming punctured, torn, ruptured, burned, crushed, squeezed, or from suffering other forms of damage or contact with hot, cold, or contaminating objects that may be encountered due to the mobility of the backpack. The bag and associated holder protect a controller from damage. Accordingly, patients undergoing IV therapy via the IV backpack may make aggressive movements without having to be mindful of damaging the IV pouch and/or the regulated controller. This feature is particularly useful for pediatric patients undergoing IV therapy who may wish to play in a park, yard, playground or other similar recreational area during a treatment session.

Further, the bag, associated holder and/or moveable flap protect the controller from weather and contamination.

Further still, because the moveable flap may be configured to obscure the pump and controls from view, patients young and old undergoing IV therapy via the IV backpack are less likely to feel intimidated, or fear treatment due to the controller connected to their body.

Additionally, the interior storage space of the bag provides a convenient location to house, store and protect additional IV pouches and/or other medical supplies.

Other benefits and advantages of the IV backpack may be noted from the following description of the invention.

### BRIEF DESCRIPTION OF THE DRAWINGS

FIG. 1 is a perspective view from the back of a portable IV backpack according to one embodiment of the invention.

FIG. 2 is a front profile view of the portable IV backpack of FIG. 1 having portions cut away to show a support rod secured inside a bag forming a part of the backpack.

FIG. 3 is profile view of the left side of the IV backpack of FIG. 1.

FIG. 4 is profile view of the right side of the IV backpack of FIG. 1.

FIG. 5 is a perspective view of a reinforcing frame for a portable IV backpack according to one embodiment.

FIG. 6 is a perspective view of another embodiment of a shield for the portable IV backpack of FIG. 1.

FIG. 7 is a perspective view of still another embodiment of the shield for the portable IV backpack of FIG. 1.

FIG. 8 is a perspective view of an embodiment of a holder for a controller used in conjunction with the portable IV backpack of FIG. 1.

FIG. 9 is a perspective view of a portable IV backpack according to a further embodiment of the invention.

### DETAILED DESCRIPTION

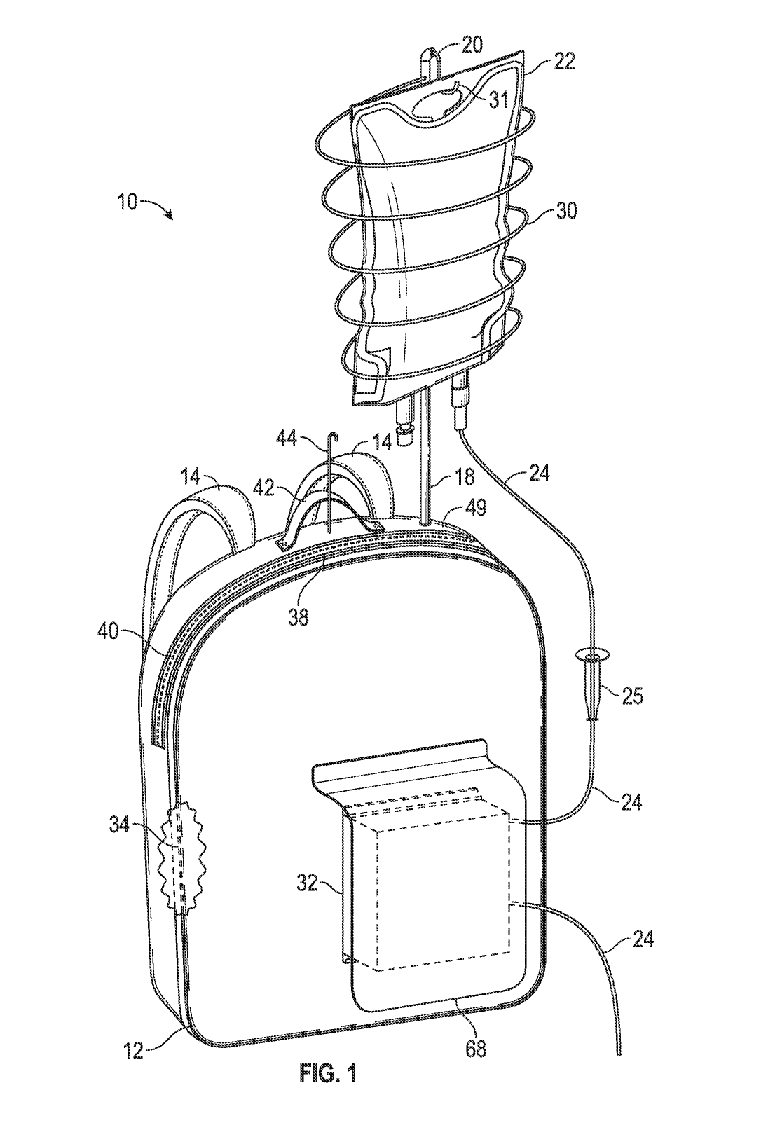

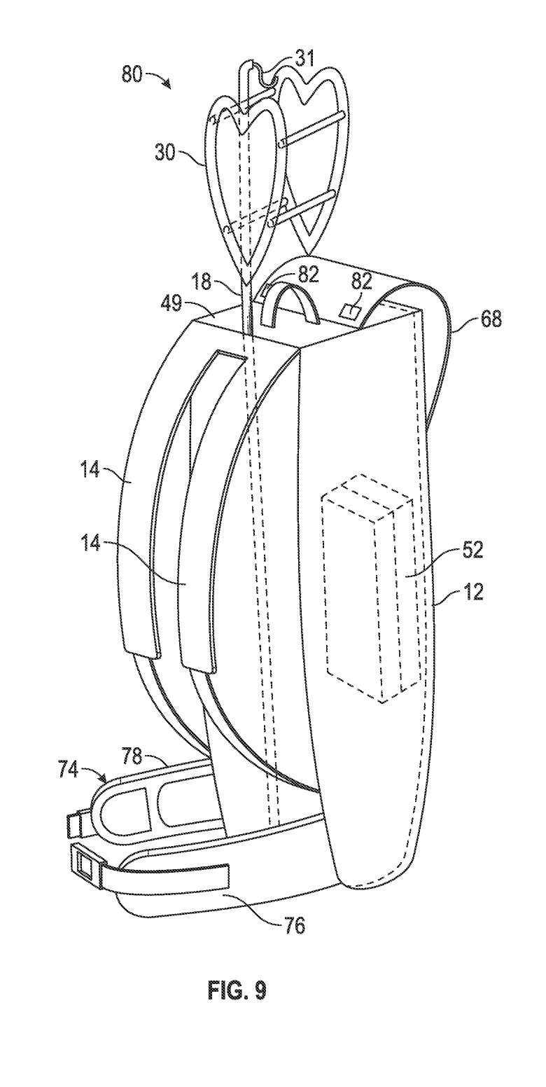

FIGS. 1-4 illustrate one embodiment of a portable IV backpack 10 of the present invention that includes a bag 12, a pair of shoulder straps 14, at least one strap 14 being necessary for carrying the backpack 10, and a support rod 18, secured to the bag 12. The support rod 18 projects upwardly above the bag 12 when the bag 12 is carried on the back of a patient (not shown). An upper end 20 of the support rod 18 is configured to support a suspended IV pouch 22 and associated tubing 24 for delivery of a medicinal fluid from the IV pouch 22 to the patient. A disconnect coupling 25 may be provided to join and separate two segments of the associated tubing 24. The IV pouch 22 is protected by a shield 30 that is connected to the upper end 20 of the support rod 18. The bag 12 also has and an associated holder 32 for a controller (shown as 50 in FIG. 8) that regulates the flow of medicinal fluid from the IV pouch 22 to the patient.

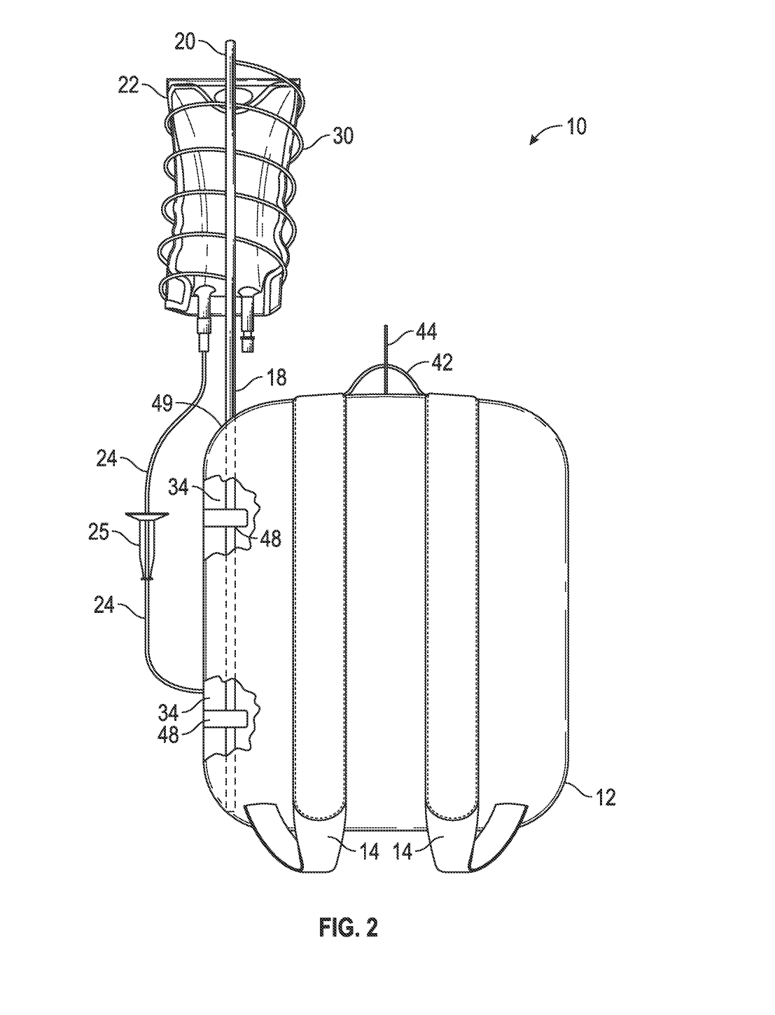

The bag 12 may made from canvas, plastic or other similar material that is sewn or thermally joined together and has an interior storage space 34 illustrated in FIGS. 1 and 2 for storage. The space is particularly useful for storing medical and other types of supplies used in IV treatment. The interior storage space 34 is accessed through an opening 38 that is opened and closed by a zipper 40, straps, or other fasteners.

The shoulder straps 14 are attached to the top and bottom of the bag 12 and permit the portable IV backpack 10 to be carried on the back of the patient in a hands free manner. The shoulder straps 14 may be adjustable to suit the size of the patient.

The bag 12 may further have a hang strap 42 and/or a hook 44 attached to the top of the bag 12 for supporting the portable IV backpack 10 when the portable IV backpack 10 is not being carried on the back of the patient.

Referring to FIG. 2, the support rod 18 is secured to the bag 12 by way of stitched fabric or other means. For example, the bag 12 may have at least one securing loop 48 attached to the bag 12 by stitching and/or adhesive. The securing loop 48 may be made of fabric, metal, plastic, wood, composite thereof, or other suitable materials. Alternatively, the bag 12 may have a fabric sleeve, in place of or in addition to the at least one securing loop 48. The support rod 18 may be made of metal, plastic, wood, composites, or other suitable materials, and extends upwardly through a hole 49 in the bag 12 to a hanger 31 at the top 20 for an IV pouch 22. The rod 18 preferably extends 18 inches above the bag 12, and provides 12 inches of clearance between the IV pouch 22 and the bag 12.

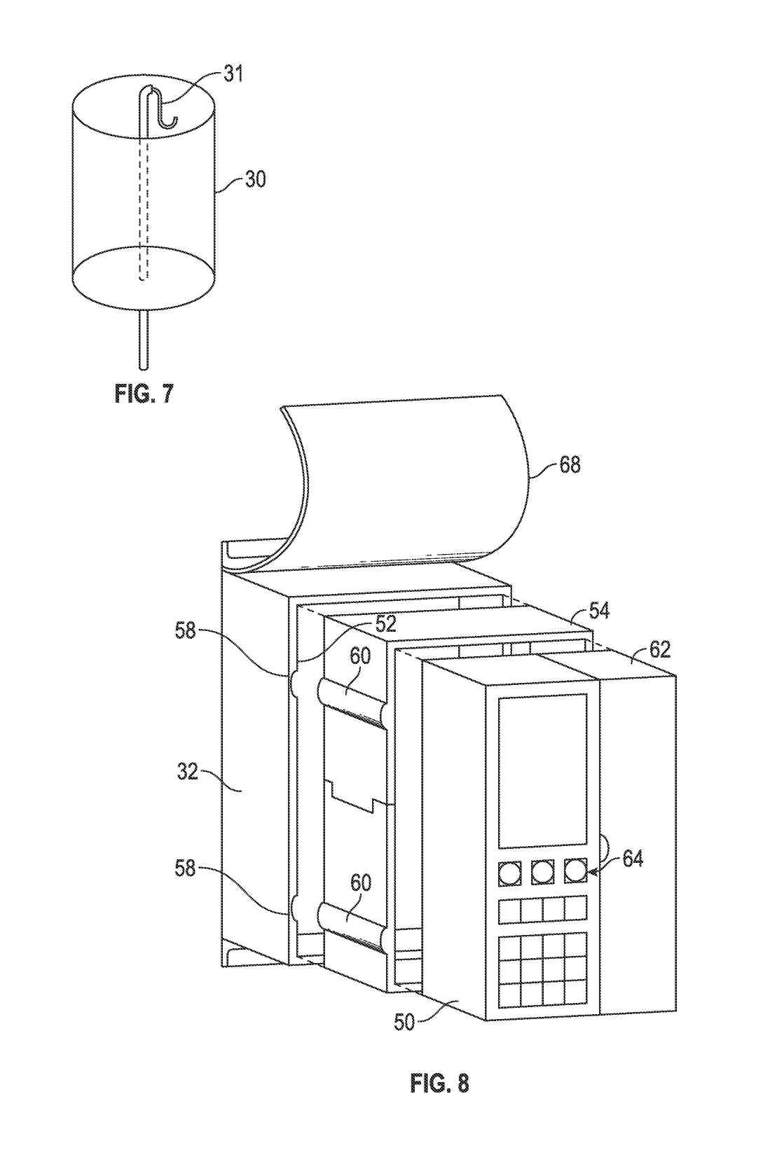

A protective shield 30 for the IV pouch 22 is connected to the top 20 of the support rod 18 and generally surrounds the IV pouch 22. The shield 30 may comprise of a series of coils, a series of loops (depicted in FIG. 6), a canister (depicted in FIG. 7), screening, or other forms of physical or thermal shielding. The shield 30 may be made of metal, plastic, wood, composite thereof or other suitable materials. The purpose of the shield 30 is to protect the IV pouch 22 and medicinal fluid inside from damage due to punctures, tears, scrapes, burns, squeezing, contamination, or other forms of damage in the event that the pouch 22 comes in contact with other objects. The shield 30 may also have an aesthetically attractive shape such as a heart (depicted in FIG. 9) for the comfort or pleasure of the patient.

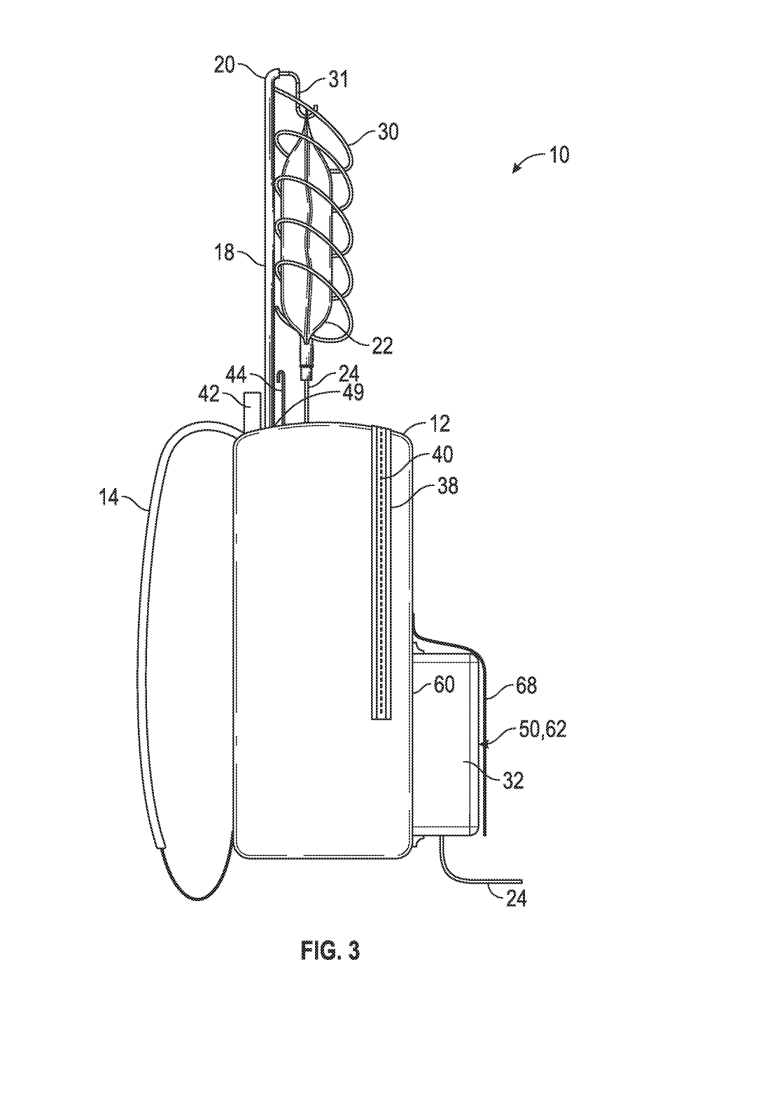

Referring to FIGS. 3 and 8, the holder 32 is a rectangular box of wood, plastic, or metal connected to the exterior of the bag 12 at the back or sides by adhesives, rivets or other fasteners and receives and secures the IV controller 50 to the bag 12. The holder 32 may have a compartment 52 houses various types of inserts 54 that are configured to receive and firmly secure corresponding controllers in the compartment 50. The compartment 52 may have internal detents 58, that interlock with corresponding protuberances 60 on the insert 54 or directly with a controller. Alternatively, the holder 32 may be as simple as a set of elastic straps that are secured to the exterior of the bag 12 and wrap around the controller 50.

FIG. 8 shows a controller 50 that is powered by a portable power supply 62, such as a battery, and has controls 64 for turning the controller 50 on and off and for regulating the delivery of medicinal fluid from the pouch (shown as 22 in FIGS. 1-4).

As shown in FIGS. 1, 3, 4, and 8, the bag 12 may also have a shielding flap 68 over the holder 32 configured to cover and protect the exposed controller 50 when the controller 50 and its controls 64 are not being accessed. The shielding flap 68 is moveable upwardly to expose the controller 50 and controls 64 for setting or monitoring, and may be secured to the bag 12 by stitching, adhesives, or one or more Velcro fasteners. The moveable flap 68 may comprise of opaque material so that the controller 50 is not visible when covered by the flap 68.

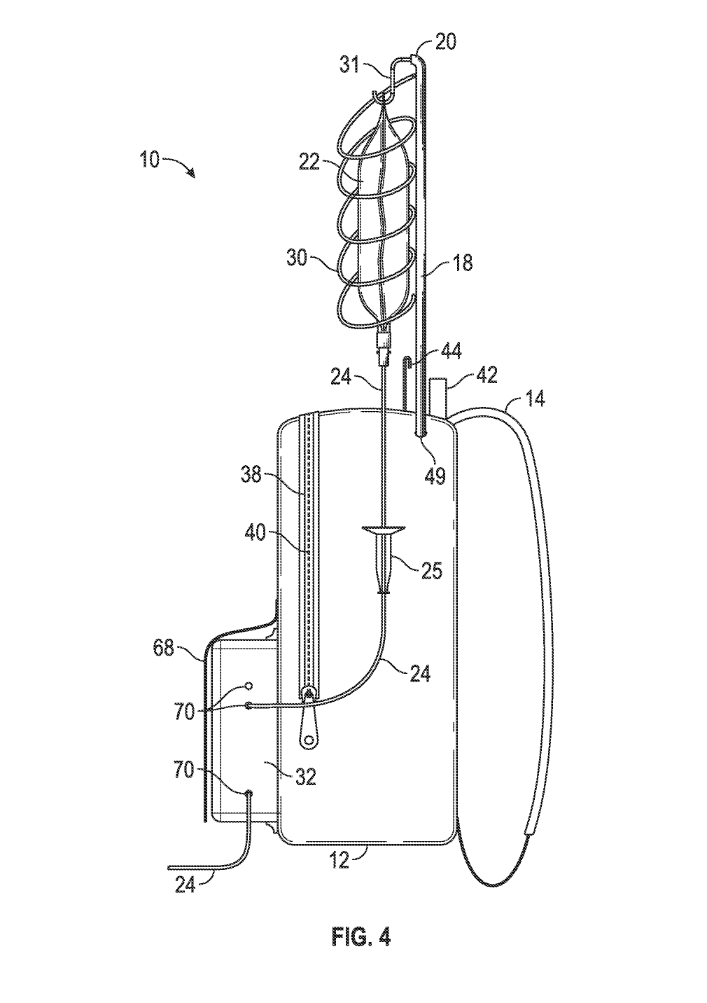

Referring to FIG. 4, the holder 32 and/or the flap 68 may have holes 70 to allow the associated tubing 24 from the pouch 22 or the patient to penetrate the holder 32 and/or flap 68 and reach the controller 50.

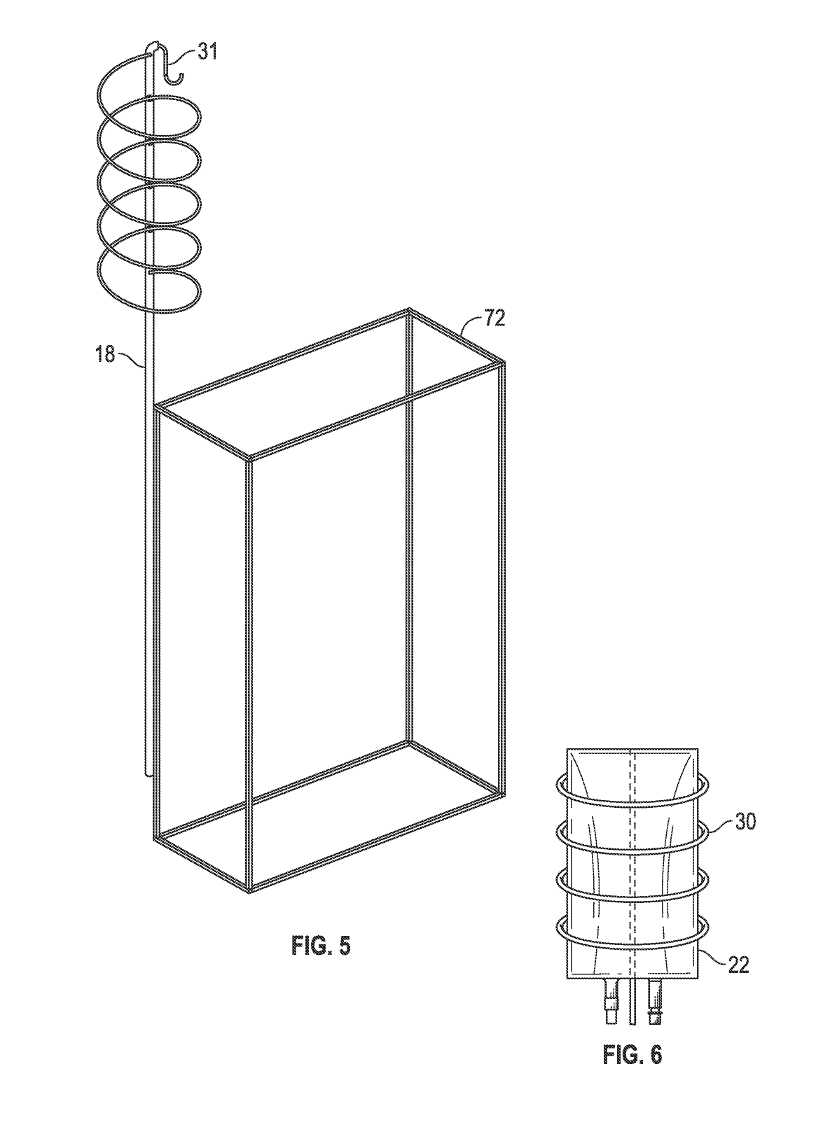

Referring to FIG. 5, the bag (shown as 12 in FIGS. 1-4) may further have a reinforcing space frame 72. The frame 72 may be contained within the interior storage space 34 of the bag, external to the bag, or integrated into the material comprising the bag. The space frame 72 may be made of rods of metal, plastic, wood, composite thereof, or other suitable materials that are joined together by welding or other suitable means.

The support rod 18 for the IV pouch 22 may be attached to or form a part of the frame 72.

FIG. 9 shows another embodiment of an IV backpack 80 according to the present invention in which parts similar to the parts of the backpack 10 of FIGS. 1-8 bear the same reference numerals. The IV backpack 80 has a waist strap 74 in addition to the pair of shoulder straps 14 to secure the backpack to the patient. The waist strap 74 may comprise a first segment 76 and a second segment 78 that can be joined by a buckle or Velcro straps.

In the backpack 80, the support rod 18 is positioned in the forward center region of the bag 12. In this position, the rod 18 may also be fastened to and form part of the internal frame (shown as 72 in FIG. 5) of the backpack 80. This positioning has the advantage of supporting the IV pouch (shown as 22 in FIGS. 1-4) at a central position where the pouch is less likely to come in contact with other objects during treatment sessions.

The shield 30 for the pouch 22 is fastened to the top of the rod 18, and is comprised of two, spaced apart, heart shaped plates for a more aesthetically pleasing appearance.

Although this invention has been shown and described with respect to the detailed embodiments thereof, it will be understood by those of ordinary skill in the art that various changes in form and detail thereof may be made without departing from the spirit and the scope of the invention.

What is claimed is:

1. A portable IV backpack compromising:
a bag having an interior storage space;
at least one shoulder strap configured to permit the backpack to be carried on the back of a patient;
a support rod secured to the bag and projecting upwardly above the bag when the backpack is carried on the back of a patient, an upper end of the projecting rod being configured to support a suspended IV pouch and associated tubing for delivery of medicinal fluid from the pouch to the patient.

2. The portable IV backpack of claim 1, wherein the bag also has an associated holder for a controller that dispenses a regulated amount of medicinal fluid from a suspended IV pouch to a patient.

3. The portable IV backpack of claim 2, wherein the associated holder exposes the controller on the exterior of the bag and renders the controller accessible for monitoring and regulation.

4. The portable IV backpack of claim 3, wherein the bag further includes a moveable flap configured to cover the exposed controller when the controller is not being accessed.

5. The portable IV backpack of claim 1, wherein the support rod is secured to the bag by way of stitched fabric.

6. The portable IV backpack of claim 1, wherein the bag further compromises at least one securing loop and the support rod is secured to the bag by way of the at least one securing loop.

7. The portable IV backpack of claim 1, wherein the bag further compromises a reinforcing space frame, and the support rod is attached to the frame.

8. The portable IV backpack of claim 7, wherein the reinforcing space frame is contained within the interior storage space of the bag.

9. The portable IV backpack of claim 1, wherein a shield is connected to the upper end of the support rod to protect a suspended IV pouch.

10. The portable IV backpack of claim 9, wherein the shield is comprised of a series of coils.

11. The portable IV backpack of claim 9, wherein the shield compromises a series of loops.

12. The portable IV backpack of claim 9, wherein the shield compromises a canister surrounding the IV pouch.

13. The portable IV backpack of claim 1, wherein the at least one shoulder strap is adjustable.

14. The portable IV backpack of claim 1, wherein the bag further compromises at least one hang strap configured to support the weight of the portable IV backpack.

15. A portable IV backpack comprising:
a bag having an interior storage space;
a pair of shoulder straps for carrying the bag and the contents of the storage space on the back of a patient;
a support rod attached to the bag and having an upper end disposed above the bag for supporting an IV pouch with medicinal fluids and associated delivery tubing; and
a shield for the IV pouch attached to the upper end of the support rod to protect the IV pouch from impact and damage.

16. A portable IV backpack as defined in claim 15, wherein the shield is comprised of a set of coils at the upper end of the support rod.

17. A portable IV backpack as defined in claim 15 wherein the shield is comprised by a canister in which an IV pouch can be placed.

18. A portable IV backpack as defined in claim 15, wherein the bag includes a holder for supporting an IV controller that regulates the flow of medicinal fluids from the IV pouch to a patient.

19. A portable IV backpack as defined in claim 18, wherein the holder for supporting an IV controller exposes the controller at the exterior of the bag for access to the controls.

20. A portable IV backpack as defined in claim 19, wherein the bag further includes a flap that covers the exposed controller and obscures the and controller from view.
